(19) United States
(12) Patent Application Publication
(10) Pub. No.: US 2011/0110980 A1
(43) Pub. Date: May 12, 2011

(54) HETERLOGOUS PRIME-BOOST IMMUNIZATION REGIMEN

(75) Inventor: David James BARTRAM, Fareham (GB)

(73) Assignee: Wyeth LLC, Madison, NJ (US)

(21) Appl. No.: 12/869,832

(22) Filed: Aug. 27, 2010

Related U.S. Application Data
(60) Provisional application No. 61/239,125, filed on Sep. 2, 2009.

(51) Int. Cl. A61K 39/15; A61P 37/04; A61P 31/14

(52) U.S. Cl. 424/215.1

(57) ABSTRACT

The invention provides methods of inducing an antigen-specific immune response in a subject by using a combination of immunogenic compositions. Generally, the method involves administering to the subject an effective amount of two different immunogenic compositions, both of which comprise bluetongue virus serotype 8.

### CROSS REFERENCE TO RELATED APPLICATIONS

This application claims priority from U.S. Provisional Application No. 61/239,125 filed Sep. 2, 2009, which is herein incorporated by reference in its entirety.

### FIELD OF THE INVENTION

The invention relates to a heterologous prime boost immunization regiment. In particular, the invention relates to a heterologous prime boost immunization to generate an antigen-specific immune response to Bluetongue Virus (BTV).

### BACKGROUND OF THE INVENTION

Bluetongue, an arthropod-borne viral disease, occurs in cattle, sheep, goats, and wild ruminants. Bluetongue lesions in affected animals resemble infectious bovine virus diarrhea, vesicular stomatitis virus, malignant catarrhal fever, mycotic stomatitis, rinderpest, photosensitization, and foot and mouth disease. Bluetongue Virus (BTV) has been incriminated as a cause of hydranencephaly in cattle and of infertility, abortion, and birth of defective young in cattle and sheep. Twenty four serotypes are reported in the literature as causing problems ranging from inapparent infection to acute fulminating infection. Chronic, persistent virus shedding cattle have also been recognized. With BTV there is a marked loss of body condition and marketing of slaughter animals may be delayed. In BTV-infected sheep, wool growth may be impaired by the development of wool breaks, which produce a defective or low yielding fleece. The marked debility following BTV infections may result in a lowering of resistance to secondary bacterial or chlamydial infections and other predatory factors. The reproductive efficiency of infected animals is also adversely affected.

Early vaccination protocols were routinely carried out in South Africa and Israel using an egg-attenuated polyvalent live virus vaccine containing a number of bluetongue strains. An egg-adapted vaccine produced by Cutter Laboratories and used in the United States has been taken off the market because of severe reactions in vaccinated sheep. Subsequently, Kemeny and Drehle, 22 AJVR 921 (1961), adapted the BTV International type 10 from eggs to bovine kidney cell cultures. This modified live virus vaccine, produced by Colorado Serum Company, is used for sheep in the United States.

The invention addresses a need in the art for an administration regimen that will produce robust cellular responses directed towards one or more antigens. In previous efforts to enhance the efficacy of immunogenic compositions, a variety of immunogenic compositions and methods have been reported using protein compositions, plasmid-based compositions, and recombinant virus constructs encoding antigens as immunogenic compositions.

Previous prime boost regimens have been used to test different combinations of heterologous viral vectors and plasmid DNA vectors. In general, the resulting cellular immune responses elicited were higher than those induced in homologous prime boost strategies (Egan et al. 2000 J. Virol., 74:7485-95, Rose et al. 2001 Cell, 106: 539-49), although any associated enhancement of protection in challenge models has not always been clear (Amara et al. 2002, J. Virol., 76:7625-31). Methods have been employed to enhance the efficacy of immunogenic compositions in eliciting an immune response specifically generated by HIV-1 proteins by administering attenuated recombinant viruses expressing one or more HIV-1 proteins. These attenuated recombinant viruses are administered in a prime-boost regimen where one recombinant virus encoding an antigen is administered prior to a second recombinant virus encoding the same antigen.

Therefore there is a need in the art for methods of enhancing the immune response to bluetongue virus vaccines.

### SUMMARY OF THE INVENTION

In one aspect, the invention provides methods for generating an antigen-specific immune response in a subject comprising administering to the subject at least one dose of a first and a second immunogenic composition comprising bluetongue virus, where the first and second immunogenic compositions are different, and where the second immunogenic composition is administered after administration of the first immunogenic composition. In some aspects of the invention, the bluetongue virus for generating an antigen-specific immune response is serotype 8. In some aspects of the invention, the first and second immunogenic compositions in the method for generating an antigen-specific immune response are selected from BOVILIS BTV-8 and ZULVAC 8 BOVIS. In some aspects of the invention, the immune response in the method for generating an antigen-specific immune response comprises an increase in antibody response to the antigen greater than that achieved by administering the first or second immunogenic compositions alone.

In some aspects of the invention, in the method for generating an antigen-specific immune response at least one of the immunogenic compositions is administered by a route selected from the group consisting of: intravenous, intradermal, subcutaneous, intramuscular, intraperitoneal, oral, rectal, intranasal, buccal, and vaginal. In a particular aspect of the invention, in the method for generating an immune response, the first immunogenic composition is administered using an intramuscular or a subcutaneous route. In a particular aspect of the invention, in the method for generating an immune response, the second immunogenic composition is administered using an intramuscular or a subcutaneous route. In some aspects of the invention, in the method for generating an antigen-specific immune response, the second immunogenic composition is administered no more than about 10 weeks after the first immunogenic composition is administered. In some aspects of the invention, in the method for generating an antigen-specific immune response, the second immunogenic composition is administered about 7 weeks after the first immunogenic composition is administered.

In one aspect, the invention provides a method of treating a viral infection in a subject comprising administering to the subject at least one dose of a first immunogenic composition comprising a bluetongue virus; followed by administering to the subject at least one dose of a second immunogenic composition comprising a bluetongue virus, where the first and second immunogenic compositions are different and wherein the second immunogenic composition is administered after administration of the first immunogenic composition. In some aspects of the invention, in the method for treating a viral infection in a subject the bluetongue virus is serotype 8. In some aspects of the invention, in the method for treating a viral infection in a subject, the immunogenic compositions are selected from BOVILIS BTV-8 and ZULVAC 8 BOVIS.

In one aspect, the invention provides an immunogenic composition for generating an antigen-specific immune response in a subject comprising first immunogenic composition comprising a bluetongue virus; and a second immunogenic composition to be administered after the first immunogenic composition, said second composition comprising a bluetongue virus. In some aspects of the invention, the immunogenic compositions for generating an antigen-specific immune response are selected from ZULVAC 8 BOVIS and BOVILIS BTV-8. In some aspects of the invention, the second immunogenic composition is administered no more than about 10 weeks after the first immunogenic composition is administered.

In one aspect, the invention provides a kit for generating an antigen specific response in a subject comprising: a first immunogenic composition comprising a bluetongue virus, and a second immunogenic composition to be administered after the first immunogenic composition comprising a bluetongue virus wherein the first immunogenic composition is different from the second immunogenic composition. In some aspects of the invention, the first and second immunogenic compositions in the kit are selected from ZULVAC 8 BOVIS and BOVILIS BTV-8.

### DETAILED DESCRIPTION

The invention provides methods of inducing an antigen-specific immune response in a subject by using a combination of immunogenic compositions. Generally, the method involves administering to the subject an effective amount of two different immunogenic compositions, both of which comprise bluetongue virus.

It has been found that administration of a priming dose of one type of bluetongue virus immunogenic composition following by a boosting dose of a different type of bluetongue virus immunogenic composition, induces in a subject an immune response including an increase in CD8+ T cell response to the antigen greater than that achieved by administering either the first or second immunogenic composition alone. This enhanced immune response appears to be a synergistic increase in cellular responses to the antigen. This combination of immunogenic compositions to bluetongue virus and order of administration generates an enhanced immune response when compared to separate administrations or compositions dosed in a different order. The invention addresses a need in the art for an administration regimen that will produce robust cellular responses directed towards one or more antigens. Currently, there is no proven method of inducing broadly neutralizing antibodies in bluetongue virus (BTV) immunogenic composition thus, an approach to increasing immunogenic composition-induced peak cellular immune responses to BTV proteins is the administration of a heterologous prime-boost combination as described.

Two commercially available vaccines prepared with bluetongue virus (BTV) serotype 8 were used. One of these vaccines is BOVILIS BTV-8, and the other vaccine is ZULVAC 8 BOVIS which is available from Fort Dodge (Southampton, United Kingdom). Immunization of animals with a single dose of BOVILIS BTV-8 vaccine elicited a measurable BTV-specific IgG response, which was significantly boosted following inoculation with ZULVAC 8 BOVIS.

The first of these two immunogenic compositions to be administered in order is referred to as the priming composition. The second of these two immunogenic compositions to be administered in order is referred to as the boosting composition. In some embodiments the priming composition is administered to the subject at least once or multiple times prior to administration of the boosting composition. Thereafter, the boosting composition is subsequently administered to the subject at least once or multiple times after at least one administration of the priming composition. Further, the invention contemplates multiple administrations of the priming composition followed by multiple administrations of the boosting composition. The term “boosting an immune response to an antigen” refers to the administration to a subject with a second, boosting immunogenic composition after the administration of the priming immunogenic composition. Administration of the boosting immunogenic composition may be performed immediately after administration of the priming immunogenic composition. Alternatively, administration of the boosting immunogenic composition may be performed about 2 to 27 weeks after administration of the priming immunogenic composition. In a preferred embodiment, the boosting immunogenic composition may be performed about seven weeks after administration of the priming immunogenic composition.

In one embodiment the present invention relates to a method of administering an immunogenic composition comprising a commercial bluetongue virus serotype 8 vaccine (BOVILIS BTV-8) followed by administration of ZULVAC 8 BOVIS, in a heterologous prime-boost combination. This combination and order of administration generates an enhanced immune response when compared to separate administrations or compositions dosed in a different order.

In certain embodiments, the immunogenic composition is capable of eliciting an immune response in a human or livestock or companion animal, such as swine, bovine, ovine, caprine, equine, deer, vicuña, canine or feline. In a preferred embodiment, the immune response elicited is a protective immune response.

An immunogenically effective amount of the vaccines of the present invention is administered to an animal in need of protection against infection with Bluetongue Virus (BTV). The immunogenically effective amount or the immunogenic amount that inoculates the animal can be easily determined or readily titrated by routine testing. An effective amount is one in which a sufficient immunological response to the vaccine is attained to protect the animal exposed to the virus. Preferably, the animal is protected to an extent in which one to all of the adverse physiological symptoms or effects of the viral disease are significantly reduced, ameliorated or totally prevented.

In one embodiment, the regimen of this invention includes those directed to the prevention and/or treatment of disease determined by the presence of a BTV antigen.

The functional outcome of vaccinating an animal against BTV can be assessed by suitable assays that monitor induction of cellular or humoral immunity or T cell activity. These assays are known to one skilled in the art, but may include measurement of cytolytic T cell activity using for example, a chromium release assay. Alternatively, T cell proliferative assays may be used as an indication of immune reactivity or lack thereof. In addition, in vivo studies can be done to assess the level of protection in an animal vaccinated against a pathogen using the methods of the present invention. Typical in vivo assays may involve vaccinating an animal with an antigen, such as the virus described herein. After waiting for a time sufficient for induction of an antibody or T cell response to occur, generally from about one to two weeks after injection, the animals will be challenged with the antigen, such as either a virus, and amelioration of one or more symptoms associated with the viral infection, or survival of the animals is monitored. A successful vaccination regimen against BTV will result in significant decrease in one or more symptoms associated with the viral infection, or a decrease in viremia, or a decrease in the number or severity of lesions associated with a viral infection, or survival when compared to the non-vaccinated controls. Serum may also be collected to monitor levels of antibodies generated in response to the vaccine injections, as measured by methods known to those skilled in the art.

In another aspect of the present disclosure, the immunogen component of an effective composition may further comprise one or more viral immunogens.

The viral immunogen may be a complete, attenuated viral immunogen that has been passaged or pre-treated to render it non-infectious and predominantly asymptomatic. Immunogens that may be employed in the generation of the compositions, including immunogenic compositions described herein, may be live, attenuated (such as CD, CAV-2, CPI, and CPV), or killed (inactivated) virions (such as CCV). If attenuated, then serial passaging of the virus using available technology may be recommended to lessen its virulence, while retaining its immunogenicity. Whole or subunit influenza virions may be inactivated by conventional means such as, for example, through chemical inactivation using one or more chemical inactivating agents including, but not limited to, one or more of binary ethyleneimine, beta-propiolactone, formalin, gluteraldehyde, and/or sodium dodecyl sulfate. Virions may also be inactivated by heat or psoralen in the presence of ultraviolet light. Methods of attenuating virulent strains of these viruses and methods of making an inactivated viral preparation are known in the art and are described in, e.g., U.S. Pat. Nos. 4,567,042 and 4,567,043. Antigens from these pathogens for use in the vaccine compositions of the present invention can be in the form of a modified live viral preparation or an inactivated viral preparation.

In other preferred embodiments, the immunogenic composition is capable of eliciting an immune response in a bovine, such as a cow, and more preferably a protective immune response.

Any of the immunogenic composition embodiments described in the instant disclosure may further have one or more pharmaceutically- or veterinary-acceptable carriers or diluents. In other embodiments, the immunogenic compositions may further comprise at least one adjuvant or at least one preservative or any combination thereof. Preferably, the immunogenic compositions according to these embodiments are a vaccine preparation.

As set forth herein, the present disclosure provides an immunogenic composition suitable for administration to a subject, such as a canine, swine, or bovine, to elicit an immune response.

As used herein “pharmaceutically acceptable or veterinary-acceptable carrier and/or diluent” includes any and all solvents, dispersion media, coatings, antibacterial and antifungal agents, isotonic and absorption delaying agents and the like. The use of such media and agents for pharmaceutically active substances is well known in the art. Supplementary active ingredients, such as antimicrobials, can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be effected by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the multivalent immunogen compositions of the present disclosure in the required amount of the appropriate solvent with various of the other ingredients enumerated herein, as required, followed by heat-sterilization, irradiation or other suitable sterilization means. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutically or veterinary acceptable carrier.

Pharmaceutical compositions comprising the immunogens of the disclosure may be manufactured by means of conventional mixing, dissolving, granulating, dragee making, levigating, emulsifying, encapsulating, entrapping or lyophilizing processes. Pharmaceutical compositions may be formulated in conventional manner using one or more physiologically acceptable carriers, diluents, excipients or auxiliaries that facilitate formulating active antimicrobial lipopeptide derivatives into preparations that can be used pharmaceutically.

Pharmaceutically acceptable carriers, diluents or excipients for therapeutic use are well known in the pharmaceutical art, and are described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992). In certain embodiments, immunogenic compositions may be formulated with a pharmaceutically or veterinary-acceptable carrier, diluent or excipient is aqueous, such as water or a mannitol solution (e.g., about 1% to about 20%), hydrophobic (e.g., oil or lipid), or a combination thereof (e.g., oil and water emulsions). In certain embodiments, any of the pharmaceutical compositions described herein have a preservative or stabilizer (e.g., an antibiotic) or are sterile.

The pharmaceutical compositions of the present disclosure can be formulated to allow the immunogens contained therein to be bioavailable upon administration of the composition to a subject. The level of immunogen in serum and other tissues after administration can be monitored by various well-established techniques, such as chromatographic or antibody (e.g., ELISA) based assays. In other embodiments, immunogenic compositions are formulated for parenteral administration to a subject in need thereof (e.g., having Gram-positive bacterial infection), such as an animal or a human. Preferred routes of administration include subcutaneous and intramuscular administrations.

Proper formulation is dependent upon the route of administration chosen, as is known in the art. For example, systemic formulations are an embodiment that includes those designed for administration by injection, e.g. subcutaneous, intravenous, intramuscular, intrathecal or intraperitoneal injection, as well as those designed for transdermal, transmucosal, oral, intranasal, or pulmonary administration. In one embodiment, the systemic formulation is sterile. In embodiments for injection, the immunogenic compositions of the instant disclosure may be formulated in aqueous solutions, preferably in physiologically compatible solutions or buffers such as Hanks's solution, Ringer's solution, mannitol solutions or physiological saline buffer. In certain embodiments, any of the immunogenic compositions described herein may contain formulatory agents, such as suspending, stabilizing or dispersing agents. Alternatively, the immunogenic compositions may be in solid (e.g., powder) form for constitution with a suitable vehicle (e.g., sterile pyrogen-free water) before use. In embodiments for transmucosal administration, penetrants, solubilizers or emollients appropriate to the barrier to be permeated may be used in the formulation. For example, 1-dodecylhexahydro-2H-azepin-2-one (Azone®), oleic acid, propylene glycol, menthol, diethyleneglycol ethoxyglycol monoethyl ether (Transcutol®), polysorbate polyethylenesorbitan monolaurate (Tween®-20), and the drug 7-chloro-1-methyl-5-phenyl-3H-1,4-benzodiazepin-2-one (Diazepam), isopropyl myristate, and other such penetrants, solubilizers or emollients generally known in the art may be used in any of the compositions of the instant disclosure. Administration can be achieved using a combination of routes, e.g., first administration using a parental route and subsequent administration using a mucosal route.

In accordance with the present disclosure, the immunogenic compositions generally include a veterinary-acceptable carrier. As described herein, a veterinary-acceptable carrier includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others. Stabilizers include albumin, among others.

In another aspect, the instant disclosure provides a pharmaceutical kit of parts comprising an immunogenic composition as defined herein. The kit may also further comprise instructions for use in the treatment of an animal disease (such as BTV, as described herein). The active agents of the immunogenic composition may be co-packaged in unit dosage form.

When administered as a liquid, vaccines may be prepared in the form of an aqueous solution, syrup, an elixir, a tincture and the like. Such formulations are known in the art and are typically prepared by dissolution of the antigen and other typical additives in the appropriate carrier or solvent systems. Suitable “physiologically acceptable” carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of porcine body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

### DEFINITIONS

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A

Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated by reference in their entirety.

The terms used herein have the meanings recognized and known to those of skill in the art, however, for convenience and completeness, particular terms and their meanings are set forth below.

The term “about” or “approximately” means within a statistically meaningful range of a value. Such a range can be within an order of magnitude, typically within 50%, more typically within 20%, more typically still within 10%, and even more typically within 5% of a given value or range. The allowable variation encompassed by the term “about” or “approximately” depends on the particular system under study, and can be readily appreciated by one of ordinary skill in the art.

“Adjuvant” means a composition comprised of one or more substances that enhances the immunogenicity of an antigen in a composition, typically a vaccine composition. An adjuvant can serve as a tissue depot that slowly releases the antigen and also as a lymphoid system activator that non-specifically enhances the immune response (Hood, et al., Immunology, Second Ed., Menlo Park, CA: Benjamin/Cummings, 1984. p. 384). Often, a primary vaccination with an antigen alone, in the absence of an adjuvant, will fail to elicit a humoral or cellular immune response. Adjuvants include, but are not limited to, complete Freund's adjuvant, incomplete Freund's adjuvant, mineral gels such as aluminum hydroxide, surface active substances such as lysolecithin, pluronic polyols, polyanions, peptides, oil or hydrocarbon emulsions, keyhole limpet hemocyanins, and potentially useful human adjuvants such as N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, BCG (bacille Calmette-Guerin) and Corynebacterium parvum. Preferably, the adjuvant is biologically acceptable. In one embodiment of the invention, the composition is administered with a combination of two adjuvants, aluminum hydroxide and saponin.

Adjuvants employed in the compositions described herein are typically “biologically acceptable adjuvants” and, thus, may be used in combination with an inactivated BTV, such that the resulting compositions may be administered in vivo without concomitant toxicity to an animal. Exemplified herein are compositions including twice inactivated BTV in combination with one or more biologically acceptable adjuvants selected from the group consisting of aluminum hydroxide, saponin, SP-Oil, SL-CD, or Carbopol. In certain embodiments, two adjuvants are used to elicit the preferred immune response to BTV. In other embodiments, a mixture of a metabolizable oil such as one or more unsaturated terpene hydrocarbon(s) may be considered for use, for example squalene or squalane, and a polyoxyethylene-polypropylene block copolymer such as Pluronic®.

An inactivated strain of BTV or molecule derived therefrom is “antigenic” when it is capable of specifically interacting with an antigen recognition molecule of the immune system, such as an immunoglobulin (antibody) or T cell antigen receptor. Typically, an antigenic molecule is a polypeptide, or variant thereof, which contains an “epitope” of at least about five and typically at least about 10 amino acids. An antigenic portion of a polypeptide, also called herein the “epitope,” can be that portion that is immunodominant for antibody or T cell receptor recognition, or it can be a portion used to generate an antibody to the molecule by conjugating the antigenic portion to a carrier polypeptide for immunization. A molecule that is antigenic need not be itself immunogenic, i.e., capable of eliciting an immune response without a carrier.

It is noted that in this disclosure, terms such as “comprises”, “comprised”, “comprising”, “contains”, “containing” and the like can have the meaning attributed to them in U.S. patent law; e.g., they can mean “includes”, “included”, “including” and the like. Terms such as “consisting essentially of” and “consists essentially of” have the meaning attributed to them in U.S. patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. patent law; namely, that these terms are closed ended.

An “immune response” to a vaccine or immunogenic composition is the development in a subject of a humoral and/or a cell-mediated immune response to molecules present in the antigen or vaccine composition of interest. For purposes of the present invention, a “humoral immune response” is an antibody-mediated immune response and involves the generation of antibodies with affinity for the antigen/vaccine of the invention, while a “cell-mediated immune response” is one mediated by T-lymphocytes and/or other white blood cells. A “cell-mediated immune response” is elicited by the presentation of antigenic epitopes in association with Class I or Class II molecules of the major histocompatibility complex (MHC). This activates antigen-specific CD4+ T helper cells or CD8+ cytotoxic T lymphocyte cells (“CTLs”). CTLs have specificity for peptide antigens that are presented in association with proteins encoded by the major histocompatibility complex (MHC) and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to restimulation with antigen. Such assays are well known in the art. See, e.g., Erickson et al., J. Immunol. (1993) 151:4189-4199; Doe et al., Eur. J. Immunol. (1994) 24:2369-2376.

The “immunogenically effective amount” is the amount of whole inactivated BTV that will elicit an immune response in an animal. This amount will depend upon the species, breed, age, size, health status of the recipient animal and will be influenced by the previous exposure of the animal to one or more strain of BTV whether that one or more strain is a virulent strain or an avirulent strain. As used herein, an “immunogenically effective amount” of whole inactivated BTV, when employed in combination with one or more suitable adjuvants, is that amount of BTV that is sufficient to enhance the immunogenicity of the BTV and thus provides for protective immunity against challenge with a pathogenic or virulent BTV strain or serotype.

The term “immunogenic” refers to the ability of an antigen or a vaccine to elicit an immune response, either humoral or cell mediated, or both. As used herein, the term “immunogenic” means that the BTV is capable of eliciting a humoral and/or cellular immune response. An immunogenic strain is also antigenic. An immunogenic composition is a composition that elicits a humoral and/or cellular immune response when administered to an animal.

The term “immunogenic composition” relates to any pharmaceutical composition containing an antigen, e.g. a microorganism, which composition can be used to elicit an immune response in an animal. The immune response can include a T cell response, a B cell response, or both a T cell and B cell response. The composition may serve to sensitize the animal by the presentation of antigen in association with MHC molecules at the cell surface. In addition, antigen-specific T-lymphocytes or antibodies can be generated to allow for the future protection of an immunized host. An “immunogenic composition” may contain a live, attenuated, or killed/inactivated vaccine comprising a whole microorganism or an immunogenic portion derived therefrom that induces either a cell-mediated (T cell) immune response or an antibody-mediated (B cell) immune response, or both, and may protect the animal from one or more symptoms associated with infection by the microorganism, or may protect the animal from death due to the infection with the microorganism.

The term “inactivated” refers to the non-infectious nature of the microorganisms to be used in a vaccine or immunogenic composition of the invention. In particular, those skilled in the art are aware of such materials that may be used to render a microorganism non-infectious for vaccine purposes, for example, BEI. In the present invention, particular methods have also been developed to render the Bluetongue virus non-infectious, but these methods have also been developed with particular emphasis on retaining the immunogenicity of the vaccine preparation, while at the same time resulting in complete inactivation of the virus preparation.

As used herein, the term “isolated” means that the referenced material is removed from its native environment. Thus, an isolated biological material can be free of some or all cellular components, i.e., components of the cells in which the native material occurs naturally (e.g., cytoplasmic or membrane component). A material is isolated if it is present in a cell extract or supernatant. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “parenteral administration” as used herein means administration by some other means than through the gastrointestinal tract, particularly to the introduction of substances into an organism by intravenous, subcutaneous, intramuscular, or intramedullary injection, but also to other non-oral and non-nasal routes of administration such as intraperitoneal injection or topical application.

The term “pathogenic” refers to the ability of any agent of infection, such as a bacterium or a virus, to cause disease. In the manner of the present invention, the term “pathogenic” refers to the ability of a Bluetongue Virus (BTV), to cause a disease in ruminants, particularly sheep or lambs. A “non-pathogenic” microorganism refers to a microorganism that lacks the characteristics noted above for the “pathogenic” strains of BTV. The disease caused by BTV is often characterized by lesions in infected animals, which resemble infectious bovine virus diarrhea, vesicular stomatitis virus, malignant catarrhal fever, mycotic stomatitis, rinderpest, photosensitization, and foot and mouth disease. Bluetongue Virus (BTV) has been incriminated as a cause of hydranencephaly in cattle and of infertility, abortion, and birth of defective young in cattle and sheep.

The term “pharmaceutically acceptable carrier” means a carrier approved by a regulatory agency of a Federal, a state government, or other regulatory agency, or listed in the U.S. Pharmacopeia or other generally recognized pharmacopeia for use in animals, including humans as well as non-human animals. The term “carrier” refers to a diluent, adjuvant, excipient, or vehicle with which the pharmaceutical composition is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. The formulation should suit the mode of administration.

The term “protecting” refers to shielding e.g. an animal, in particular, a mammal, for example, a sheep, a lamb, a goat or a cow, from infection or a disease, by inducing an immune response to a particular pathogen, e.g. Bluetongue Virus. Such protection is generally achieved following treating an animal with the vaccine compositions described herein.

The term “purified” as used herein refers to material that has been isolated under conditions that reduce or eliminate the presence of unrelated materials, i.e., contaminants, including native materials from which the material is obtained. For example, a purified bacteria or protein is typically substantially free of host cell or culture components, including tissue culture or egg proteins, non-specific pathogens, and the like. As used herein, the term “substantially free” is used operationally, in the context of analytical testing of the material. Typically, purified material substantially free of contaminants is at least 50% pure; more typically at least 90% pure, and more typically still at least 99% pure. Purity can be evaluated by chromatography, gel electrophoresis, immunoassay, composition analysis, biological assay, and other methods known in the art. Methods for purification are well known in the art. The term “substantially pure” indicates the highest degree of purity, which can be achieved using conventional purification techniques known in the art.

“Saponins” are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Hemolysis of erythrocytes is an example of this phenomenon, which is a property of certain, but not all, saponins. Saponins are known as adjuvants in vaccines for systemic administration. The adjuvant and haemolytic activity of individual saponins has been extensively studied in the art (Lacaille-Dubois and Wagner, supra). For example, “Quil A” (derived from the bark of the South American tree Quillaja Saponaria Molina), and fractions thereof, are described in U.S. Pat. No. 5,057,540 and “Saponins as vaccine adjuvants”, Kensil, C. R., Crit Rev Ther Drug Carrier Syst, 1996, 12 (1-2):1-55, and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1). These structures have been reported to have adjuvant activity (EP 0 109 942 B1; WO 96/11711). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1. Also described in these references is the use of QS7 (a non-haemolytic fraction of Quil-A) which acts as a potent adjuvant for systemic vaccines. Use of QS21 is further described in Kensil et al. (1991, J. Immunology vol 146, 431-437). Combinations of QS21 and polysorbate or cyclodextrin are also known (WO 99/10008). Particulate adjuvant systems comprising fractions of Quil-A, such as QS21 and QS7 are described in WO 96/33739 and WO 96/11711. Other saponins which have been used in systemic vaccination studies include those derived from other plant species such as Gypsophila and Saponaria (Bomford et al., Vaccine, 10(9):572-577, 1992). Saponins are also known to have been used in mucosally applied vaccine studies, which have met with variable success in the induction of immune responses. Quil-A saponin has previously been shown to have no effect on the induction of an immune response when antigen is administered intranasally (Gizurarson et al. 1994 Vaccine Research 3, 23-29). Whilst, other authors have used this adjuvant with success (Maharaj et al., Can. J. Microbiol, 1986, 32(5):414-20. Chavali and Campbell, Immunobiology, 174(3):347-59). ISCOMs comprising Quil A saponin have been used in intragastric and intransal vaccine formulations and exhibited adjuvant activity (Mcl Mowat et al., 1991, Immunology, 72, 317-322; Mcl Mowat and Donachie, Immunology Today, 12, 383-385). QS21, the non-toxic fraction of Quil A, has also been described as an oral or intranasal adjuvant (Sumino et al., J. Virol., 1998, 72(6):4931-9, WO 98/56415). The use of other saponins in intranasal vaccination studies has been described. For example, Chenopodium quinoa saponins has been used in both intranasal and intragastric vaccines (Estrada et al., Comp. Immunol. Microbiol. Infect. Dis., 1998, 21(3):225-36).

The term “SL-CD” refers to a sulpholipo-cyclodextrin that falls within the family of cyclodextrin adjuvants described in U.S. Pat. Nos. 6,610,310 and 6,165,995. Typically, SL-CD is formulated in a mixture with a metabolizable oil such as one or more unsaturated terpene hydrocarbons, for example, squalane and preferably with a non-ionic surfactant, such as polyoxyethylene sorbitan monooleate.

The term “SP-Oil” refers to an adjuvant that is an oil emulsion comprising: 1% to 3% vol/vol of polyoxyethylene-polyoxypropylene block copolymer; 2% to 6% vol/vol of squalane; 0.1% to 0.5% vol/vol of polyoxyethylene sorbitan monooleate; and a buffered salt solution.

The term “Mammals” include monotremes (e.g., platypus), marsupials (e.g., kangaroo), and placentals, which include livestock (domestic animals raised for food, milk, or fiber such as hogs, sheep, cattle, and horses) and companion animals (e.g., dogs, cats). “Ungulates” include, but are not limited to, cattle (bovine animals), water buffalo, bison, sheep, swine, deer, elephants, and yaks. Each of these includes both adult and developing forms (e.g., calves, piglets, lambs, etc.). The immunogenic composition of the invention can be administered either to adults or developing mammals, preferably livestock. The term “ruminant” refers to any variety of hoofed, even footed, and usually horned mammals that characteristically have their stomachs divided into four sections, including cows, sheep, giraffes, goats and deer.

As used herein, “treatment” (including variations thereof, for example, “treat” or “treated”) refers to any one or more of the following: (i) the prevention of infection or reinfection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided which treat, including prophylactically and/or therapeutically immunize, a host animal against a viral infection. The methods of the present invention are useful for conferring prophylactic and/or therapeutic immunity to an animal, preferably a mammal, such as a sheep, lamb, cow or goat. The methods of the present invention can also be practiced on mammals for biomedical research applications.

The terms “vaccine” or “vaccine composition”, which are used interchangeably, refer to pharmaceutical compositions comprising at least one immunogenic composition that induces an immune response in an animal. A vaccine or vaccine composition may protect the animal from disease or possible death due to an infection, and may or may not include one or more additional components that enhance the immunological activity of the active component. A vaccine or vaccine composition may additionally comprise further components typical to pharmaceutical compositions. A vaccine or vaccine composition may additionally comprise further components typical to vaccines or vaccine compositions, including, for example, an adjuvant or an immunomodulator. The immunogenically active component of a vaccine may comprise complete live organisms in either their original form, or as attenuated organisms in a modified live vaccine, or organisms inactivated by appropriate methods in a killed or inactivated vaccine, or subunit vaccines comprising one or more immunogenic components of the virus, or genetically engineered, mutated or cloned vaccines prepared by methods known to those skilled in the art. A vaccine or vaccine composition may comprise one or simultaneously more than one of the elements described above.

### EXAMPLES

### Example 1

To evaluate the neutralizing antibody response to a single booster dose of ZULVAC 8 BOVIS (Fort Dodge) in cattle primed with a commercial BTV-8 vaccine (BOVILIS BTV-8) containing ≧500 antigenic units/ml prior to inactivation. Three commercial inactivated BTV-8 vaccines are authorized for use in cattle in the UK.

Twenty-four steers primed with two doses of BTV-8 vaccine seven months previously, were divided into three groups with similar BTV-8 serum neutralization test (SNT) antibody titres. All were BTV-8 RT-PCR negative. Group 1 (n=4) remained untreated as controls. Group 2 (n=10) received a single dose of BTV-8 vaccine. Group 3 (n=10) received a single dose of ZULVAC 8 BOVIS. Blood samples were collected at weekly intervals post-vaccination for 8 weeks and again at 16 weeks and sent to an independent international reference laboratory in the UK for SNT.

BOVILIS BTV-8 is a commercially available BTV vaccine. A 1 ml dose of this vaccine contains:

At least 500 Antigenic Units/ml of Bluetongue Virus Serotype 8 prior to inactivation.

Adjuvants per dose: 16.7 mg of 100% Aluminum hydroxide; 0.31 mg saponin.

The composition also comprises trometamol; sodium chloride, maleic acid, antifoam, water for injection.

ZULVAC 8 BOVIS is a BTV vaccine available from Fort Dodge. A 2 ml dose contains inactivated Bluetongue Virus, serotype 8, strain BTV-8/BEL2006/02 titre ≧10^{6.7} TCID₅₀^{*} (^{*}measured before inactivation).

Adjuvants per dose: 385.2 mg (4 mg Al³⁺) of 3% aluminium hydroxide gel and 0.4 mg Saponins.

The composition also comprises:

Aluminum gel hydroxide

Saponines

Thiomersal

Potassium chloride

Potassium dihydrogen phosphate

Disodium hydrogen phosphate dodecahydrate

Sodium chloride

### Water for injections

### RESULTS

SNT antibody titres were expressed as log₁₀ reciprocal of the highest positive serum dilution. Area under the curve (AUC) was used as a summary indicator of the repeated measures. Mean AUC differed significantly across Groups 1 to 3 (one-way ANOVA, p=0.023): 173.0 vs. 191.8 vs. 272.5 units² respectively. Post hoc LSD tests indicated statistically significant differences between Groups 2 and 3 (p=0.017) and 1 and 3 (p=0.025) but not between Groups 1 and 2 (p=0.651). Pairwise differences were not significant after Bonferroni adjustment.

### CONCLUSIONS

In cattle primed with BTV-8 vaccine, a booster dose of ZULVAC 8 BOVIS produced a five-fold greater neutralizing antibody response than a booster dose of BTV-8 vaccine. Challenge studies are required to determine differences in protection from infection.

What is claimed:

1. A method for generating an antigen-specific immune response in a subject comprising
administering to the subject at least one dose of a first immunogenic composition comprising a bluetongue virus;
administering to the subject at least one dose of a second immunogenic composition comprising a bluetongue virus,
wherein the first and second immunogenic compositions are different, and wherein the second immunogenic composition is administered after administration of the first immunogenic composition.

2. The method of claim 1 wherein the bluetongue virus is serotype 8.

3. The method of claim 2 wherein the first immunogenic composition is BOVILIS BTV-8 and the second immunogenic composition is ZULVAC 8 BOVIS.

4. The method of claim 2 wherein the first immunogenic composition is ZULVAC 8 BOVIS and the second immunogenic composition is BOVILIS BTV-8.

5. The method of claim 1 wherein at least one of the first and second immunogenic compositions is administered by a route selected from the group consisting of: intravenous, intradermal, subcutaneous, intramuscular, intraperitoneal, oral, rectal, intranasal, buccal, and vaginal.

6. The method of claim 5, wherein the first immunogenic composition is administered using an intramuscular or a subcutaneous route.

7. The method according to claim 6 wherein the second immunogenic composition is administered using an intramuscular or a subcutaneous route.

8. The method of claim 1 wherein the second immunogenic composition is administered no more than about 10 weeks after the first immunogenic composition is administered.

9. The method of claim 8 wherein the second immunogenic composition is administered about 7 weeks after the first immunogenic composition is administered.

10. A method of treating a viral infection in a subject comprising:
administering to the subject at least one dose of a first immunogenic composition comprising a bluetongue virus;
administering to the subject at least one dose of a second immunogenic composition comprising a bluetongue virus, and
wherein the first and second immunogenic compositions are different, and wherein the second immunogenic composition is administered after administration of the first immunogenic composition.

11. The method of claim 10 wherein the bluetongue virus is serotype 8.

12. The method of claim 11 wherein the first immunogenic composition is BOVILIS BTV-8 and the second immunogenic composition is ZULVAC 8 BOVIS.

13. The method of claim 12 wherein the first immunogenic composition is ZULVAC 8 BOVIS and the second immunogenic composition is BOVILIS BTV-8.

14. An immunogenic composition for generating an antigen-specific immune response in a subject comprising:
a first immunogenic composition comprising a bluetongue virus; and
a second immunogenic composition to be administered after the first immunogenic composition, said second composition comprising a bluetongue virus.

15. The immunogenic composition of claim 14, wherein the first and second immunogenic compositions are selected from ZULVAC 8 BOVIS and BOVILIS BTV-8.

16. A kit for generating an antigen specific response in a subject comprising: a first immunogenic composition comprising a bluetongue virus, and a second immunogenic composition to be administered after the first immunogenic composition comprising a bluetongue virus wherein the first immunogenic composition is different from the second immunogenic composition

17. The kit of claim 16, wherein the first and second immunogenic compositions are selected from ZULVAC 8 BOVIS and BOVILIS BTV-8.
